(19) United States
(12) Patent Application Publication
(10) Pub. No.: US 2014/0316258 A1
(43) Pub. Date: Oct. 23, 2014

(54) MULTIPLE SECTION PET WITH ADJUSTABLE AUXILIARY SECTION

(71) Applicants: Guenter Hahn, Barrington, IL (US); James Frank Caruba, Bartlett, IL (US)

(72) Inventors: Guenter Hahn, Barrington, IL (US); James Frank Caruba, Bartlett, IL (US)

(73) Assignee: SIEMENS MEDICAL SOLUTIONS USA, INC., Malvern, PA (US)

(21) Appl. No.: 13/868,256

(22) Filed: Apr. 23, 2013

(51) Int. Cl. A61B 6/03; A61B 6/00

(52) U.S. Cl. CPC A61B 6/037; A61B 6/4417
USPC 600/427; 600/425

(57) ABSTRACT

A system includes a gantry, a first positron emission tomography (PET) section including a first detector ring oriented about an axis, and a second PET section supported by the gantry and including a second detector ring oriented about the axis. The gantry is adjustable to move the second PET section relative to the first PET section.

### BACKGROUND

The present embodiments relate to positron emission tomography (PET).

Nuclear medicine uses radiation emission to acquire images that show the function and physiology of organs, bones or tissues of the body. Radiopharmaceuticals are introduced into the body by injection or ingestion. These radiopharmaceuticals are attracted to specific organs, bones, or tissues of interest. The radiopharmaceuticals cause gamma photons to emanate from the body, which are then captured by a detector. The interaction of the gamma photons with a scintillation crystal of the detector produces a flash of light. The light is detected by an array of optical sensors of the detector.

Positron emission tomography (PET) is a nuclear medicine imaging technique that uses a positron emitting radionuclide. PET is based on coincidence detection of two gamma photons produced from positron-electron annihilation. The two gamma photons travel in generally opposite directions from the annihilation site, and can be detected by two opposing detectors of a ring of detectors. Annihilation events are typically identified by a time coincidence in the detection of the two gamma photons. The opposing detectors identify a line-of-response (LOR) along which the annihilation event occurred.

PET may be combined with another imaging modality in a multimodality system. Such multimodality imaging systems may have diagnostic value. PET-computed tomography (CT) multimodality imaging systems allow scans to be performed back-to-back or in a same coordinate system and with similar timing. The axial fields of view of the individual modalities are typically as close together as possible in order to minimize the impact of patient motion and increase spatial correlation of the respective data sets. PET-CT and multimodality systems commonly combine the benefits of a high local resolution modality (e.g., CT imaging) with a modality with high functional sensitivity (e.g., PET) to spatially align detailed anatomy and functional information.

### SUMMARY

By way of introduction, the embodiments described below include systems and methods of imaging using multiple positron emission tomography (PET) sections. The position of at least one of the PET sections is adjustable to provide correlated imaging of spaced apart portions of a subject and/or imaging of a larger field of view than that provided by the PET sections individually.

In a first aspect, a system includes a gantry, a PET section including a first detector ring oriented about an axis, and a second PET section supported by the gantry and including a second detector ring oriented about the axis. The gantry is adjustable to move the second PET section relative to the first PET section.

In a second aspect, a method of imaging with first and second PET sections including first and second detector rings, respectively, includes adjusting an axial position of the second PET section along an axial direction relative to the first PET section, and receiving scan data via the first and second detector rings concurrently.

In a third aspect, a system includes a first PET section including at least a first group of first detectors, a second PET section including at least a second group of second detectors, a positioner configured to adjust a position of the second PET section along an axial direction relative to the first PET section, and a data acquisition system coupled to the first and second PET sections to process signals from the first and second PET sections.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of a PET-CT imaging system having multiple PET sections in accordance with one embodiment.

FIG. 2 is a block diagram of PET subsystem equipment of the PET-CT imaging system of FIG. 1 in accordance with one embodiment.

FIG. 3 is a flow diagram of a method of configuring and operating a PET-CT imaging system having multiple PET sections in accordance with one embodiment.

### DETAILED DESCRIPTION OF THE PRESENTLY PREFERRED EMBODIMENTS

Systems and methods with multiple positron emission tomography (PET) sections are provided for whole body or other imaging. The disclosed embodiments may achieve whole body and other imaging by partitioning PET-related scanning into multiple fields of view separated along an axis about which detector rings of the PET sections are oriented. As described below, one or more PET fields of view are movable or adjustable in the axial direction. In some embodiments, an auxiliary PET field of view is spatially and independently deployed relative to a fixed PET field of view. Two or more separated regions of a subject may thus be imaged at the same time. Through concurrent imaging of spaced apart regions, the PET imaging may support dynamic studies that correlate various body functions. For example, the dynamic study may correlate the cardiopulmonary function of the heart and lungs with the neurological function of the brain. The tissues and functions involved in the dynamic studies may vary.

The disclosed methods and systems may be applied in the context of hybrid imaging modalities, such as those that combine PET scanning with x-ray computed tomography (CT). Increasing the axial field of view (FOV) of the PET subsystem presents challenges for whole body and other imaging due to the high channel count of the PET subsystem and/or the high cost of scintillation materials (e.g., LSO crystals), sensors, and/or acquisition electronics used in the PET subsystem. The axial FOV of commercially available PET scanners often falls well short of extending over the entire length of a subject to be scanned. For instance, the axial FOV of such scanners may fall in the range of about 16 cm to about 22 cm. The disclosed methods and systems may be applied in the context of PET-only imaging modalities.

Instead of trying to increase the axial FOV to support full body imaging, the disclosed methods and systems may address these challenges by supporting PET scanning over selected regions of the body. Such selected or zoned scanning may be achieved via an adjustable gantry configured to move an auxiliary PET section in an axial direction relative to, e.g., a fixed PET section. The separation distance between the selected regions may thus vary. For example, the adjustment provided by the disclosed methods and systems may allow the PET sections to be disposed adjacent to one another to scan adjacent (e.g., contiguous) regions of the subject. Such adjacent placement of the PET sections may alternatively or additionally be used to increase the sensitivity of the PET subsystem.

The disclosed methods and systems may use or include a gantry that supports a primary PET section and a secondary or auxiliary PET section. The gantry may be configured such that the primary PET section is fixed, while the auxiliary PET section may be movable. Although described below in connection with examples having a pair of PET sections, the number of primary (e.g., fixed) and/or auxiliary (e.g., movable) PET sections may vary. The construction and other characteristics of the gantry may also vary. For example, the disclosed systems may include multiple gantries to support the fixed and movable PET sections. The multiple gantries may be coupled to one another and/or integrated with one another to any desired extent. All of the PET sections may be movable (i.e., none of the PET sections are fixed).

The disclosed methods may include a positioner or other mechanical unit to drive the PET section to a desired axial position. A gantry that supports the movable PET section may be coupled to the positioner for axial or other movement along one or more tracks, rails, or other structures.

In some embodiments, the axial position of the auxiliary PET section is indexed or referenced to the position of the primary PET section via a calibration procedure. An alignment or other calibration phantom may be positioned during the calibration procedure within both fields of view as described below. Alternatively or additionally, the patient table may be used and/or configured to calibrate the axial positioning. The primary and auxiliary PET sections may be spatially correlated in one or more ways.

The primary and auxiliary PET sections may share or use a common data acquisition system, a common time base, and/or common synchronization signals. The scan data generated by the primary and auxiliary PET sections may thus be correlated in time within the data acquisition system and other processing.

Although described below in the context of a PET-CT hybrid modality system, the disclosed systems and methods are not limited to use with any particular type of planning subsystem. Scan data used for planning the PET scan(s) and/or rendering the PET scan data (and/or other purposes) may be acquired via a variety of different types of scanners (e.g., projection, emission, magnetic resonance, etc.). For example, the planning or support modality need not include a CT scanner, and may include or involve any now or hereafter developed imaging technology. The planning or support modality need not include or involve tomography.

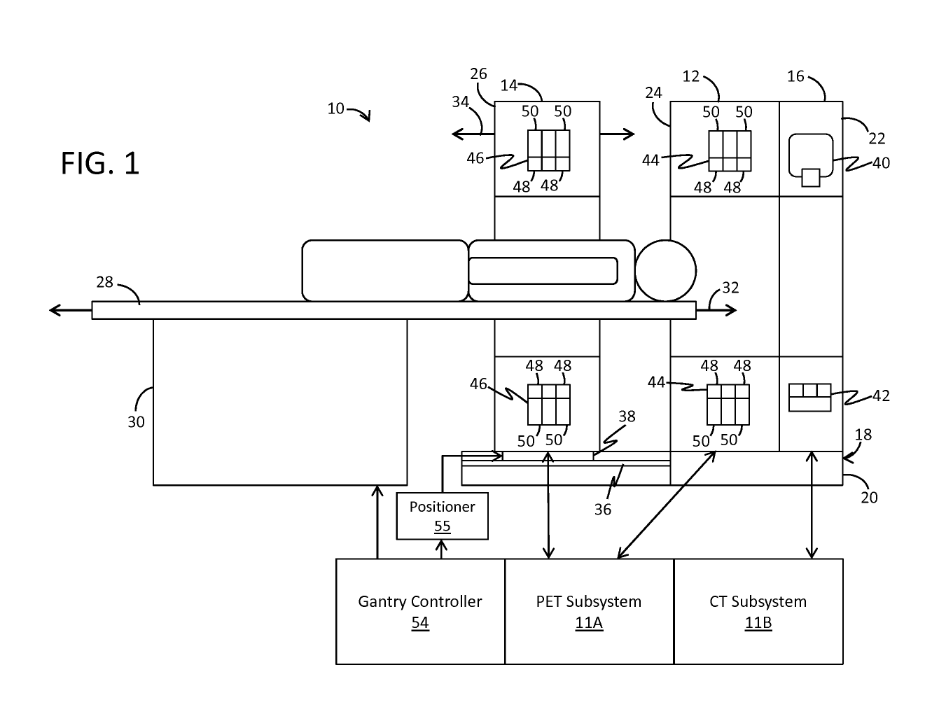

FIG. 1 shows a hybrid positron emission tomography (PET) and x-ray computed tomography (CT) system 10. The hybrid PET-CT system 10 includes a PET subsystem 11A and a CT subsystem 11B. The PET subsystem 11A includes or is coupled to a primary PET section 12 and an auxiliary PET section 14. The primary PET section 12 is stationary or fixed, while the auxiliary PET section 14 is movable relative to the primary PET section 12, as described above. The primary PET section 12 may be movable in some embodiments. The primary PET section 12 and the auxiliary PET section 14 include respective scanners as described below.

The primary and auxiliary PET sections 12, 14 may be similarly configured or have one or more characteristics that differ. For example, the primary and auxiliary PET sections 12, 14 may have fields of view that are equal in length or different. The physical pixel size of the detector block in the primary and secondary PET sections 12, 14 may also be equal, integer multiples of one another, or fractions of one another. For example, it may be useful to configure one of the PET sections with a higher resolution (e.g., for brain scans), in which case the pixel sizes may be selected for the primary and auxiliary PET sections so that a number of the smaller pixels may be combined to effectively form one of the larger pixels of the PET section with the lower resolution, which may be useful, for instance, in scans in which the PET sections are placed together. For example, four pixels having a pixel size of 2 mm×2 mm may be combined to form a single 4 mm×4 mm pixel (i.e., a four to one pixel ratio). Other pixel sizes and ratios may be used. The fractional relationship of the pixel sizes is determinative of the number of pixels to be used.

The CT subsystem 11B includes or is coupled to a CT scanner 16. In this example, the CT scanner 16 is disposed adjacent the primary PET section 12. The CT scanner 16 and the primary PET section 12 may be enclosed in a common housing or cover. The CT scanner 16 may be disposed along an outward or distal side of the primary PET section 12. The position of the CT scanner 16 may vary. For example, the CT scanner 16 may be disposed between the primary and auxiliary PET sections 12, 14. The construction, configuration, and other characteristics of the CT scanner 16 may vary. For example, the CT scanner 16 may include a C-arm unit.

Additional, different, or fewer components may be provided. For example, the PET-CT system 10 may include one or more additional scanners. Additional PET or CT sections or units may be incorporated. The system 10 may include additional or alternative imaging modalities or scanning equipment. For example, the system 10 may include a fluoroscopy projection unit.

The PET sections 12, 14 and the CT scanner 16 are structurally supported by a gantry 18. The gantry 18 may include a framework of structural components to support the operation of the PET and CT subsystems 11A, 11B. For instance, one or more structural components may support the movement of the auxiliary PET section 14, as described below. In this example, the gantry 18 includes a base 20, a CT gantry section 22, a primary PET gantry section 24, and an auxiliary PET gantry section 26.

The CT gantry section 22, the primary PET gantry section 24, and the auxiliary PET gantry section 26 include respective frames that define corresponding tubular openings or bores or other openings within which a subject is positioned during scanning operation. The shape and size of the bores need not be similar as shown in FIG. 1. For example, the CT scanner 16 may have a laterally open examination subject bore or any other opening defining a field of view. The subject rests upon a bed, gurney, or other platform 28 supported by a table base 30. The platform 28 is movable in an axial or longitudinal direction 32 through the bores. The movement of the platform 28 may be driven independently of the movement of the gantry 18. Alternatively, the platform movement may be facilitated by one or more components of the gantry 18.

The gantry 18 is adjustable to move the auxiliary PET section 14 along or in an axial direction 34 relative to the primary PET section 12. The axial directions 32 and 34 may be parallel as shown. In some embodiments, the gantry 18 includes one or more tracks or rails 36 to support the movement and axial adjustment. In this example, the track(s) 36 are embedded or disposed within the base 20 of the gantry 18. The auxiliary PET section 14 may be driven along the track(s) 36 via one or more couplings 38. The configuration of the track(s) 36 may vary. For example, the track(s) 36 may be alternatively or additionally disposed on scaffolding and/or other structures of the gantry 18 (e.g., above the bores, such as along a ceiling). The construction, configuration, and other characteristics of the coupling(s) 38 may vary accordingly.

A number of structural components of the gantry 18 may be directed to support other movement occurring during operation. For example, the gantry 18 may be configured to support the rotational movement of an x-ray source 40 and a detector 42 of the CT scanner 16. The x-ray source 40 and the detector 42 may rotate within a housing or other cover or enclosure of the CT gantry section 22. The x-ray source 40 and the detector 42 may rotate about a longitudinal axis centered within the bores and corresponding with, or parallel to, the axial directions 32, 34. The CT gantry section 22 and the primary PET gantry section 24 may share a common enclosure. The CT gantry section 22 and the primary gantry section 24 need not be adjacent or oriented relative to one another as shown.

The primary and auxiliary PET sections 12, 14 include respective detector rings 44, 46. The detector rings 44, 46 may be oriented about the longitudinal axis around which the components of the CT scanner 16 rotate. The primary and auxiliary PET gantry sections 24, 26 are configured to support the detector rings 44, 46, respectively. In this example, the primary PET gantry section 24 of the gantry 18 supports the primary PET section 12 in a manner that the axial position of the detector ring 44 is fixed. In contrast, the auxiliary PET gantry section 26 is configured to adjust the axial or other position of the detector ring 46 of the auxiliary PET section 14. Different zones or regions of a subject lying on the table platform 28 may thus be scanned by the auxiliary PET section 14 as a result of the adjustment.

The detector rings 44, 46 may include respective sets of detector blocks disposed in a ring arrangement. For example, each detector ring 44, 46 may include a set of 48 detector blocks. Each detector block may, in turn, include a number of scintillation crystals 48 and optical detectors or sensors 50. The scintillation crystals 48 and optical sensors 50 may be arranged in a two-dimensional array within each detector block. For example, each detector block in the detector ring 44 of the primary PET section 12 may have a 13×13 array of scintillation crystals (e.g., 4 mm crystals). The optical sensors 50 may include photomultiplier tubes, silicon avalanche photodiodes (APDs), or other photosensors configured to detect the optical light created by the scintillation crystals 48. The photons generated by each crystal array are captured by a number of the optical sensors 50 (e.g., four photomultiplier tubes per block) in each detector block. The scintillation crystals 48 may include bismuth germanium oxide, gadolinium oxyorthosilicate, or lutetium oxyorthosilicate crystals, but other crystals may be used.

In nuclear medicine imaging, such as PET, radioactive tracer isotopes, or radiopharmaceuticals, are taken internally, for example intravenously or orally. As the radioisotope undergoes positron emission decay (also known as positive beta decay), the radioisotope emits a positron, an antiparticle of the electron with opposite charge. The emitted positron travels in tissue for a short distance, during which time the positron loses kinetic energy, until the positron decelerates to a point where the positron interacts with an electron. The encounter annihilates both electron and positron, producing a pair of annihilation (gamma) photons moving in approximately opposite directions. These events are detected when the gamma radiation reaches one of the scintillation crystals 48 in the detector ring 44, 46, creating a burst of light, which is detected by the optical detector(s) 50 in the detector block. The detector rings 44, 46 thus capture data representing the radiation emitted, directly or indirectly, by the radiopharmaceuticals. The PET subsystems 11A, 11B form images from the captured data.

The axial width of the detector blocks in the detector rings 44, 46 may establish a respective field of view (FOV) of the primary and auxiliary PET sections 12, 14. FIG. 1 schematically shows the axial width of the detector rings 44, 46. In this example, the detector blocks of the detector ring 44 include three detector blocks along the axial width of the detector ring 44, thereby establishing a corresponding axial FOV. The auxiliary PET section 14 may thus have the same or a different axial FOV. In this example, the detector blocks of the detector ring 46 also include three detector blocks along the axial width of the detector ring 46, thereby establishing the same axial FOV for the auxiliary PET section 14. The relative FOV sizes may vary. For example, the auxiliary PET section 14 may have a larger FOV than the primary PET section 12.

In some embodiments, the gantry 18 is configured such that the detector ring 46 is movable into a position along the longitudinal axis in which the detector rings 44 and 46 are disposed adjacent one another. The primary and auxiliary PET gantry sections 24, 26 may be configured to allow the outer pixels (e.g., the scintillation crystals 48 and/or the optical detectors 50) of the detector rings 44, 46 to be adjacent to one another. For example, the detector rings 44, 46 may be positioned within the primary and auxiliary PET gantry sections 24, 26 and otherwise configured such that minimal to no axial space or gap is present between the detector rings 44, 46 when the auxiliary PET gantry section 26 is moved as close as possible to the primary PET gantry section 24. For instance, axial ends or faces of the primary and auxiliary PET gantry sections 24, 26 may be configured such that minimal or no gap is present between the outer detector pixels and an outer cover or housing of the primary and auxiliary PET gantry sections 24, 26. Separate housings may be used, allowing the primary and auxiliary PET gantry sections 24, 26 within a same cover or housing to be positioned adjacent to each other. With the outer pixels of the detector rings 44, 46 adjacent to one another, the PET subsystem of the system 10 may be configured for single field of view (FOV) operation. A gap may be provided while adjacent but still allowing use as a single FOV.

The system 10 includes a gantry controller 54 to control the axial positioning of the auxiliary PET gantry section 26. The gantry controller 54 may be configured to direct a number of drive units of the gantry 18 to translate and/or rotate various system components. For example, the gantry controller 54 may direct a positioner 55 or other drive unit to move the auxiliary PET gantry section 26 along the track(s) 36 to position the auxiliary PET section 14 in the axial direction.

The gantry controller 54 and/or the positioner 55 may be configured to control the spacing between the detector rings 44, 46. In some cases, the gantry controller 54 and the positioner 55 are configured to adjust the spacing discretely rather than continuously, e.g., in accordance with a step function. The discrete steps may correspond with the axial width of a detector pixel in the detector rings 44, 46. For example, the positioner 55 may limit or control the spacing between the respective ends of the detector rings 44, 46 to distances corresponding with an integer multiple of the axial width of the detector pixels. The distance or spacing may be measured between the crystal planes of the ends of the detector rings 44, 46. Non-integer spacing may be used.

The gantry 18, the gantry controller 54, and/or the positioner 55 may be configured to ensure or maintain crystal plane co-planarity of the PET sections. Alternatively or additionally, the lack of co-planarity of the PET sections may be addressed through dynamic compensation. Such compensation may be directed to the physical position and/or to correcting the scan data acquired via the secondary PET section. An open loop or closed loop control system may be used to provide dynamic compensation. For example, the gantry controller 54 or the positioner 55 may include a shaft encoder to detect the position of a shaft of a motor driving one or more gears configured to drive the movement of the PET section. Alternatively or additionally, the gantry controller 54 or the positioner 55 may include an encoder (e.g., a linear encoder) to measure the position of the PET section directly. These control system components may be used to compensate for lack of planarity of the movement of the PET section and/or other errors that may vary with the position of the PET section along an axis of movement. Such compensation may be useful to maintain co-planarity of the PET sections.

In other embodiments, gantry control functionality may be provided separately by the PET and CT subsystems 11A, 11B. Such functionality may be provided by the PET and CT subsystems 11A, 11B to any desired extent. Alternatively or additionally, control of the axial positioning of the auxiliary PET gantry section 26 is provided separately from other gantry control functions, such as table position.

Other gantry arrangements or assemblies may be used. For example, the system 10 may include more than one gantry. A separate or discrete gantry may be provided for each scanner, section, or modality. The gantry 18 may include additional, fewer, or alternative components. For example, the gantry 18 may include scaffolding or other support structures above the detector rings 44, 46 and/or the CT scanner 16.

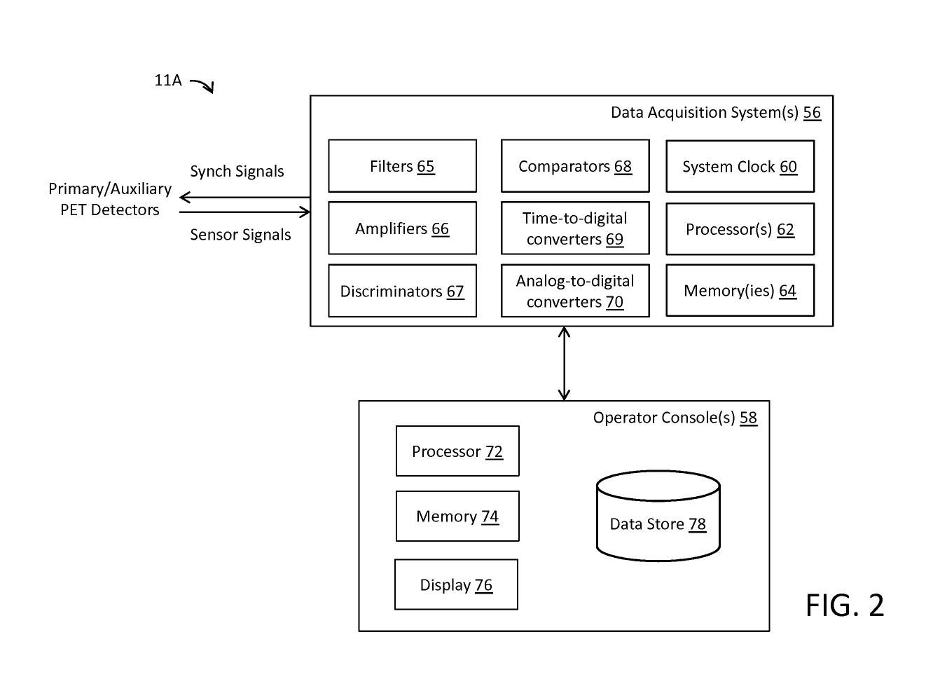

FIG. 2 shows processing equipment of the PET subsystem 11A of the system 10 in greater detail. Some equipment, such as scanning and gantry equipment, of the PET subsystem 11A is not shown. The processing equipment may include a number of components for controlling the operation of the primary and secondary PET sections 12, 14, and/or for gathering, receiving, processing, rendering, and otherwise using or handling the signals generated by the primary and secondary PET sections 12, 14. The components may be directed to a variety of functions, including, for instance, power supply, control and other data communication, image data processing, system clock signals, and cooling. Some of the components may support one or more functions of the CT scanner 16.

In this example, the PET subsystem 11A includes a data acquisition system 56 and an operator console 58. The data acquisition system 56 may be configured to receive, convert, and otherwise process the signals generated by the scanning. The operator console 58 may be configured to direct or control the operation of the scanning components of the system 10 (FIG. 1), reconstruct images from the scan data, store data representative of the images, and display the images. The data acquisition system 56 (and/or other subsystem components) may also be configured to control or otherwise support the scanning by providing power supply, timing, and other functionality. In some cases, the data acquisition system 56 and the operator console 58 may provide these and other functions for both the PET and CT subsystems 11A, 11B. In other embodiments, the PET and CT subsystems 11A, 11B are supported separately. The scanning operation of the PET and CT subsystems 11A, 11B may be controlled and supported via the data acquisition system 56, the operator console 58, and/or other subsystem equipment using any known or hereafter developed techniques, as supplemented and/or otherwise modified as described herein.

The data acquisition system 56 may be coupled to both the primary and auxiliary PET sections 12, 14 (FIG. 1) to process and support the generation of signals from both of the primary and auxiliary PET sections 12, 14. For example, the data acquisition system 56 may include a system clock 60 configured to provide a common time base for the signals generated by the detector rings 44, 46 (FIG. 1). The system clock 60 may be used in connection with digital sampling of the signals generated by the PET sections 12, 14, as well as the CT scanner 16. The system clock 60 may be used to generate common synchronization signals provided to both of the primary and auxiliary PET sections 12, 14. The common synchronization signals may be used to temporally correlate operation of the detector rings 44, 46. The synchronization signals may be analog or digital. The common time base may be provided via circuitry other than the system clock 60, and/or otherwise generated or maintained by the data acquisition system 56 or other circuitry of the PET subsystem 11A.

The data acquisition system 56 may include a variety of digital and analog electronic circuits in addition to the system clock 60 to support the operation of the primary and auxiliary PET sections 12, 14. In this example, the digital electronic circuit(s) includes a processor 62 and a memory 64 coupled to the processor 62. The memory 64 may store a number of computer-readable instruction sets to be implemented by the processor 62 on data representative of the scan data. For example, the processor 62 may be configured by the instruction sets to control the position and/or movement of the auxiliary PET detector(s). In another example, the instructions may be configured to cause the processor 62 to assign time stamps to the scan data for later use in image reconstruction. The digital electronics of the data acquisition system 56 may include additional or alternative components. For example, the data acquisition system 56 may include an additional processor or other digital circuit configured to generating or providing a digital clock signal. The data acquisition system 56 may include respective processors, memories, and/or other digital circuitry to support implementation of the PET data processing and/or control tasks.

The analog electronics of the data acquisition system 56 may be directed to receiving analog output or other sensor signals provided by the detector rings 44, 46 (FIG. 1). In this example, the analog electronic circuitry includes front-end filters 65, amplifiers 66, discriminators 67, comparators 68, time-to-digital convertors 69, and analog-to-digital converters 70. The filters 65 and the amplifiers 66 may be configured to receive and condition the sensor signals provided by the detector rings 44, 46 prior to processing by the discriminators 67 and the comparators 68 and conversion into the digital domain by the time-to-digital converters 69 and the analog-to-digital converters 70. Additional, fewer, or alternative analog circuitry may be provided to support the processing of the sensor signals. Additional or alternative signal processing may be provided by the digital electronics. The data acquisition system 56 may include additional circuitry for other functions, such as power supply.

In the embodiment of FIG. 2, the operator console 58 provides both data processing and user interface functions for the PET subsystem 11A (and, in some cases, the CT subsystem). The data processing and user interface functions may be provided separately in other embodiments. For example, image reconstruction and other processing of the scan data may be implemented by a separate computing system. The PET and CT subsystems may be supported by respective operator consoles and/or computing systems. The operator console 58 may be in communication with the gantry controller 54 (FIG. 1) and the data acquisition system 56 to control the operation of the PET-CT system 10. The gantry controller 54 may be integrated with the operator console 58 to any desired extent. In this example, the operator console 58 is configured to process PET and CT scan data provided by the data acquisition system 56. In other embodiments, a separate computing system may be provided for such processing.

The operator console 58 includes a processor 72, as well as a memory 74 and a display 76 coupled to the processor 72. The processor 72 may be configured to implement one or more data processing routines on the scan data provided by the data acquisition system 56. For example, the data processing routines may be configured to implement image reconstruction and other procedures to support the rendering of representations of the scan data on the display 76. The scan data may be stored in a database or other data store 78. The data store 78 may be configured to store the scan data (and/or other data) at one or more stages of processing.

The data processing routines implemented by the processor 72 may also be directed to correction of the scan data. The correction(s) may be useful to address distortion introduced by the adjustability and/or positioning of the auxiliary PET section 14. For example, the processor 72 may be configured to correct the scan data captured by the detector ring 46 of the auxiliary PET section 14 based on axial position calibration data. The scan data may reflect an offset from a desired positioning of the detector ring 46 in any one or more of the dimensions of the auxiliary PET section 14. The offset may arise from inaccuracies introduced by the positioner 55 or other component of the system 10.

In some embodiments, scan data may be obtained before the subject is scanned to calibrate the correction procedures implemented by the processor 72. Such calibration may be directed to a particular axial positioning of the auxiliary PET section 14 and/or to a set of axial positions. For example, the calibration data may be representative of a calibration phantom that extends across the fields of view of both detector rings 44, 46. In one embodiment, the calibration phantom presents a spatial frequency or other pattern that varies as a function of axial position. Other types of calibration phantoms or techniques may be used to correct for axial position of the detector ring 46. The corrections for axial positioning may be applied to the scan data for the detector ring 46 before image reconstruction and other processing. Vector-based, offset-based, and other calibration techniques may be used. In some cases, the positioner 55 may establish a sufficiently precise axial position for the auxiliary PET section 14, in which case such axial position calibration need not be implemented.

Alternative or additional scan data corrections may be implemented. For example, the processor 72 may be configured to correct the scan data captured by the detector ring 46 to compensate for crystal planarity of the detector rings 44, 46 (FIG. 1). The scintillation crystals 48 (FIG. 1) of the detector rings 44, 46 may not be co-planar as a result of the movement of the auxiliary PET section 14. The lack of co-planarity may be addressed via the scan data corrections.

Scan data provided by the CT scanner 16 may be used for planning the scanning procedures to be implemented by the primary and auxiliary PET sections 12, 14. The scan data from the CT scanner 16 may also be used during image reconstruction, as an overlay, or for other purposes (e.g., registration and/or attenuation correction).

The data acquisition system 56, the operator console 58 and other subsystem equipment may include any number of respective processors configured to control and communicate with the scanning components of the PET-CT system 10 (FIG. 1). The data acquisition system 56 may include, for instance, a coincidence processor for the PET subsystem. Other processors may be configured to provide acquisition control for the CT and PET subsystems or PET image reconstruction.

Any now known or later developed PET imaging system components may be used in connection with the functionality described herein. The electronics of the PET subsystem 11A may include additional equipment. For example, the PET subsystem 11A may include power supply equipment.

Each memory 64, 74 or data store 78 is a buffer, cache, RAM, removable media, hard drive, magnetic, optical, database, or other now known or later developed memory. Each memory 64, 74 or data store 78 is a single device or group of multiple devices. Each memory 64, 74 or data store 78 is shown within the PET subsystem 11A, but may be outside or remote from other components of the PET subsystem 11A, such as a database or PACS memory.

Each memory 64, 74 or data store 78 may store data at different stages of processing. For example, the memory 74 may store raw data representing detected events without further processing, filtered or thresholded data prior to reconstruction, reconstructed data, filtered reconstruction data, an image to be displayed, an already displayed image, or other data. Each memory 64, 74 or data store 78 (or a different memory) may store data used for processing, such as storing the data after one or more iterations and prior to a final iteration in reconstruction. For processing, the data bypasses the memory 74, is temporarily stored in the memory 74, or is loaded from the memory 74.

Each memory 64, 74 is additionally or alternatively a non-transitory computer readable storage medium storing processing instructions. For example, the memory 74 stores data representing instructions executable by the programmed processor 72 for reconstructing a positron emission tomography image for dynamic study and/or reconstructing an image in emission tomography. As another example, the memory 64 stores data representing instructions executable by the programmed processor 62. The instructions for implementing the processes, methods and/or techniques discussed herein are provided on non-transitory computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software stored or otherwise embodied on a computer-readable memory, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

Each processor 62, 72 is a general processor, digital signal processor, graphics processing unit, application specific integrated circuit, field programmable gate array, digital circuit, combinations thereof, or other now known or later developed device for processing emission information. Each processor 62, 72 is a single device, a plurality of devices, or a network. For more than one device, parallel or sequential division of processing may be used. Different devices making up each processor 62, 72 may perform different functions, such as one processor for filtering and/or subtracting raw data or reconstructed images. Each processor 62, 72 may include an application specific integrated circuit or field programmable gate array for performing various operations, such as iterative reconstruction. In one embodiment, the processor 72 is a control processor or other processor of a PET imaging system. The processor 72 may be a processor of a computer or workstation.

Each processor 62, 72 operates pursuant to stored instructions to perform various acts described herein. For example, the processor 72 may be operable to process data indicative of detected events, correct for axial positioning, and implement iterative reconstructions from different collections of data. Each processor 62, 72 may be configured by code or instructions sets stored on a memory, by firmware, and/or by hardware to perform any or all of the acts described herein.

The display 76 is a CRT, LCD, plasma screen, projector, printer, or other output device for showing images generated by the PET subsystem 11A. The display 76 may be used to display a user interface for controlling the PET subsystem 11A and/or CT subsystem 11B. The CT subsystem 11B may have a separate display or user interface for control thereof. The display 76 may alternatively or additionally be used to display the PET images generated by the disclosed systems and methods. Such PET images may include separately rendered or reconstructed images from the respective PET sections and/or include an image with an extended FOV when the PET sections are adjacent.

The above-described PET and CT processing equipment may be integrated to any desired extent. For example, the system 10 may include separate data acquisition systems and/or separate operator consoles to control and/or operate the PET and CT subsystems.

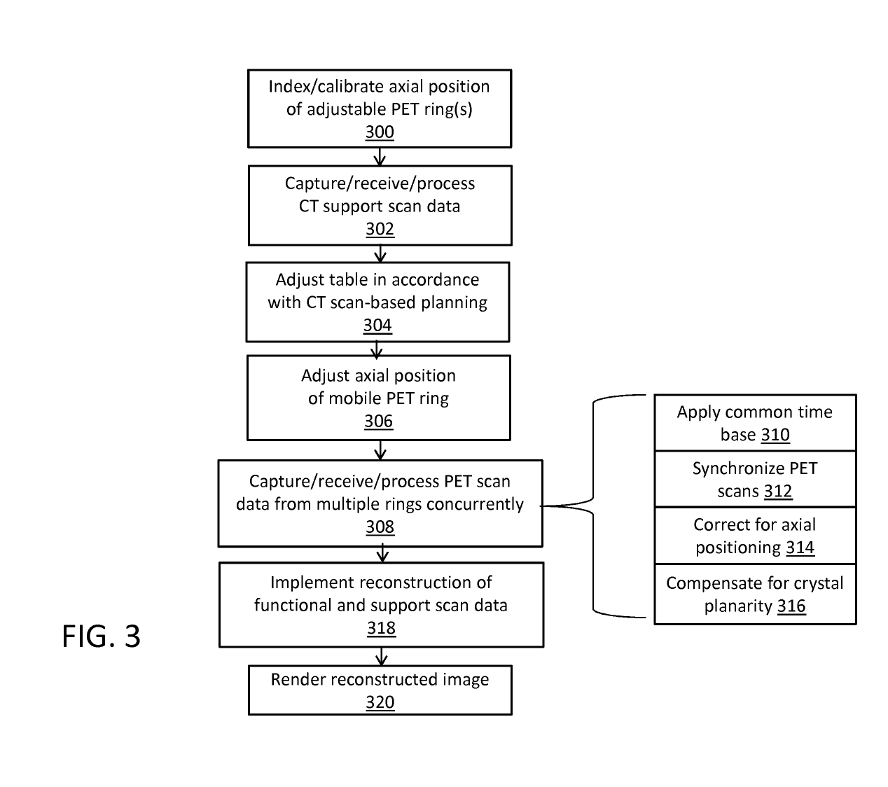

FIG. 3 shows one embodiment of a method for imaging with a system having multiple PET sections, such as the primary and auxiliary PET sections of the PET-CT system 10 described above. The method is performed in the order shown, but other orders may be used. For example, calibration for one or more axial positions of an auxiliary PET section may be implemented after CT scan data is obtained and/or after a subject bed is repositioned in accordance with planning (which may be based on such CT scan data). Additional, fewer, or alternative acts may be implemented. For example, an image reconstructed from the scan data need not be rendered. As another example, calibration is assumed or was previously performed so is not used in a given procedure.

The method may begin in an act 300 in which calibration of an auxiliary PET section is implemented. The calibration may include an indexing procedure in which a detector ring of the auxiliary PET section is moved through a set of axial positions at which scan data of, e.g., a phantom, is obtained. The calibration may also include generation and storage of correction data in a memory based on such scan data, as described above. The calibration may be directed to correcting for axial position and/or crystal planarity, each of which may be relative to a stationary or primary PET section.

In the embodiment of FIG. 3, CT scan data is received in an act 302. The CT scan data may be used to support subsequent PET scanning. For example, the CT scan data may be used to position a subject bed or table in an act 304 and/or adjust the axial position of the auxiliary PET section (relative to the primary PET section) in an act 306. Movement of the auxiliary PET section to a desired axial position may thus be based on the CT scan data. Through such adjustments, multiple regions or zones of a subject may be scanned via the primary and auxiliary PET sections in an act 308. PET scan data may be captured or received from the primary and auxiliary PET sections concurrently. Such concurrent scanning may be useful temporally correlating multiple functions in the subject.

The scan data may then be processed as described above. In this example, the reception or processing of the scan data includes the application of a common time base to the detector rings of the primary and auxiliary PET sections in an act 310. The operation of the detector rings of the primary and auxiliary PET sections may thus be temporally synchronized in an act 312. For example, a common synchronization signal may be distributed to the detector rings of the primary and auxiliary PET sections.

The processing of the scan data may include the implementation of one or more data correction procedures. In this example, the scan data captured by the detector ring of the auxiliary PET section is corrected based on axial position calibration data in an act 314. The correction may be implemented by, for instance, one of the above-described processors and/or another processor. The scan data correction may be based the calibration or correction data obtained via the act 300. For example, the calibration or correction data may be representative of an alignment phantom positioned within both respective fields of view of the detector rings (when docked together) or indexed into the fields of view (when separate). Such calibration data (or other calibration data) may alternatively or additionally be used to correct the scan data to compensate for crystal planarity of the detector rings in an act 316.

After the scan data is corrected, reconstruction and other data processing may be implemented in an act 318 to generate an image from the scan data. Such processing may be implemented by one of the above-described processors and/or another processor. The reconstructed image may then be rendered via the above-described display or stored in a memory in an act 320.

The above-described imaging method may be implemented with a PET gantry that supports the deployment of a primary PET field of view (FOV) and a secondary PET FOV spatially indexed or referenced to the primary FOV. The primary PET FOV may be disposed in a fixed axial position, while the secondary PET FOV may be movable in the axial direction relative to the primary PET FOV. The movement may support axial positions ranging from one in which, e.g., the secondary PET FOV is positioned adjacent to the primary PET FOV (non-spaced apart positioning) to spaced apart axial positioning having a gap between the primary and secondary fields of view that corresponds with an integer multiple of a single block detector pixel width. Any size gap may result, such as a gap exceeding the FOV of one or both sections. The gap may be indexed in accordance with the distance between the end crystal planes of the primary and secondary PET fields of view.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A system comprising:
a gantry;
a first positron emission tomography (PET) section comprising a first detector ring oriented about an axis; and
a second PET section supported by the gantry and comprising a second detector ring oriented about the axis;
wherein the gantry is adjustable to move the second PET section along the axis relative to the first PET section.

2. The system of claim 1, further comprising a data acquisition system coupled to the first and second PET sections to process signals from the first and second PET sections, wherein the data acquisition system is configured with a common time base for the signals generated by the first and second PET rings.

3. The system of claim 1, further comprising a data acquisition system coupled to the first and second PET sections to process signals from the first and second PET sections, wherein the data acquisition system is configured to provide common synchronization signals to temporally correlate operation of the first and second detector rings.

4. The system of claim 1, further comprising a processor coupled to the first and second PET sections and configured to correct scan data captured by the second detector ring based on axial position calibration data.

5. The system of claim 1, further comprising a processor coupled to the first and second PET sections and configured to correct scan data captured by the second detector ring to compensate for crystal planarity of the first and second detector rings.

6. The system of claim 1, wherein the first and second PET rings comprise a respective number of detector blocks disposed in a ring arrangement, the detector blocks of the first and second PET rings establishing differently sized fields of view for the first and second PET rings.

7. The system of claim 1, further comprising a positioner configured to control a spacing between respective end crystal planes of the first and second detector rings to distances corresponding with an integer multiple of an axial width of a pixel of the first detector ring or the second detector ring.

8. The system of claim 1, wherein the gantry supports the first PET section such that an axial position of the first detector ring is fixed.

9. The system of claim 1, wherein the gantry is configured such that the second detector ring is movable into a position along the axis in which the first and second detector rings are disposed adjacent to one another for single field of view (FOV) operation.

10. The system of claim 1, wherein the gantry is adjustable to move the second PET section along the axis.

11. A method of imaging with first and second positron emission tomography (PET) sections comprising first and second detector rings, respectively, the method comprising:
adjusting an axial position of the second PET section along an axial direction relative to the first PET section; and
receiving scan data via the first and second detector rings concurrently.

12. The method of claim 11, wherein receiving the scan data comprises applying a common time base to the first and second detector rings.

13. The method of claim 11, wherein receiving the scan data comprises temporally synchronizing operation of the first and second detector rings with a common synchronization signal.

14. The method of claim 11, wherein receiving the scan data comprises correcting, with a processor, scan data captured by the second detector ring based on axial position calibration data.

15. The method of claim 11, wherein receiving the scan data comprises correcting, with a processor, scan data captured by the second detector ring to compensate for crystal planarity of the first and second detector rings.

16. The method of claim 11, further comprising receiving x-ray computed tomography (CT) scan data, wherein adjusting the axial position of the second PET section comprises moving the second PET section to an axial position based on the x-ray CT scan data.

17. The method of claim 11, further comprising correcting, with a processor, the scan data received via the second detector ring based on calibration data representative of an alignment phantom positioned within both respective fields of view of the first and second PET rings.

18. A system comprising:
a first positron emission tomography (PET) section comprising at least a first group of first detectors;
a second PET section comprising at least a second group of second detectors;
a positioner configured to adjust a position of the second PET section along an axial direction relative to the first PET section; and
a data acquisition system coupled to the first and second PET sections to process signals from the first and second PET sections.

19. The system of claim 18, wherein the positioner is further configured to establish a spacing between respective end crystal planes of the first and second detectors that corresponds with an integer multiple of an axial width of a pixel of the first group of the first detectors or the second group of the second detectors.

20. The system of claim 18, wherein:
the data acquisition system is configured with a common time base for the signals generated by the first and second PET sections; and
the data acquisition system is further configured to provide common synchronization signals to temporally correlate operation of the first and second detectors.
